(19) United States
(12) Patent Application Publication
(10) Pub. No.: US 2002/0156444 A1
(43) Pub. Date: Oct. 24, 2002

(54) DISPOSABLE DIAPER

(76) Inventor: TOSHIFUMI OTSUBO, EHIME-KEN (JP)

Correspondence Address: LOWE, HAUPTMAN, GOPSTEIN, GILMAN & BERNER, LLP, 1700 DIAGONAL ROAD, SUITE 310, ALEXANDRIA, VA, 22314, US

(21) Appl. No.: 08/937,515

(22) Filed: Sep. 25, 1997

(30) Foreign Application Priority Data
Sep. 30, 1996 (JP) 8-259571

(51) Int. Cl. A61F 13/15; A61F 13/20

(52) U.S. Cl. 604/385.3; 604/366; 604/385.22; 604/396

(57) ABSTRACT

A disposable diaper includes a backsheet consisting essentially of an elastic inner layer and an inelastic outer layer made of fibrous material. The inelastic outer layer is joined to the elastic inner layer in its stretched state at a plurality of spots for joining intermittently arranged on the elastic inner layer in its stretched state. In each of areas enclosed by a plurality of intersecting lines each defined by a plurality of the spots for joining closely arranged in succession, the inelastic outer layer is free to float off from the elastic inner layer and thereupon to form a plurality of pleats as the elastic inner layer is made free to contact.

### BACKGROUND OF THE INVENTION

This invention relates to a disposable diaper and more particularly to such a diaper having a plurality of pleats formed by a backsheet thereof.

It is well known in the art of disposable diapers to make a backsheet thereof from an inner layer of plastic film and an outer layer of nonwoven fabric in order to improve a touch of the diaper. For example, Japanese Laid-Open Utility Model Application No. Hei3-122824 discloses a disposable diaper comprising a backsheet consisting of an elastic outer layer of plastic film or rubber film and an elastic inner layer of nonwoven fabric intermittently joined to each other so that these layers may be elastic for stretch and contraction in the form of a single member.

However, the nonwoven fabric as the elastic outer layer making a constituent of the known backsheet may often lose a softness peculiar to nonwoven fabrics once it have been joined to the plastic film as the elastic inner layer making another constituent of the backsheet. Consequently, a soft touch required for the diaper is apt to be deteriorated in comparison with the case in which the nonwoven fabric is used independently for the backsheet. Such known backsheet is disadvantageous also from an economical viewpoint, since the elastic nonwoven fabric and film are generally more expensive than those which are inelastic.

### SUMMARY OF THE INVENTION

In view of the problems as has been described above, it is a principal object of the invention to provide a disposable diaper having a backsheet having a desired softness as well as an elasticity and being obtainable at a relatively low cost.

The object set forth above is achieved, according to the invention, by a disposable diaper having a front waist region, a rear waist region and a crotch region therebetween, said diaper comprising a liquid-permeable topsheet, a liquid-impermeable backsheet and a liquid-absorbent core disposed therebetween, wherein:

said backsheet in at least said front waist region comprises an elastic inner layer and an inelastic outer layer and overlapped with said elastic inner sheet;

and said elastic inner layer and said inelastic outer layer are joined to each other at a plurality of intermittently arranged spots, with said elastic inner layer being stretched transversely of said diaper so that, in areas enclosed by intersecting lines each described by a plurality of said spots closely arranged in succession, said inelastic outer layer is free to float off from said elastic inner layer and thereupon to form a plurality of pleats as said elastic inner layer is made free to contract transversely of said diaper.

The disposable diaper according to the invention is characterized by the backsheet at least in the front waist region comprising the elastic inner layer and the inelastic outer layer. The inelastic outer layer floats off from the elastic inner layer as the latter is contracted so as to form a plurality of pleats being convex outward. Such pleats formed by the inelastic outer layer effectively provides the diaper with a comfortable soft touch peculiar to the nonwoven fabric.

Other and further objects, features and advantages of the invention will appear more fully from the following description.

### BRIEF DESCRIPTION OF THE DRAWINGS

FIG. 1 is a perspective view showing an embodiment of the disposable diaper according to the invention as partially broken away;

FIG. 2 is a diagram showing a part of a backsheet of the diaper;

FIG. 3 is a sectional view taken along a line III-III in FIG. 2;

FIG. 4 is a fragmentary plan view showing the backsheet as being in its stretched state;

FIG. 5 is a fragmentary sectional view showing the backsheet as being in its stretched state; and

FIG. 6 is a view similar to FIG. 3 showing a variant of the backsheet.

### DESCRIPTION OF THE PREFERRED EMBODIMENT

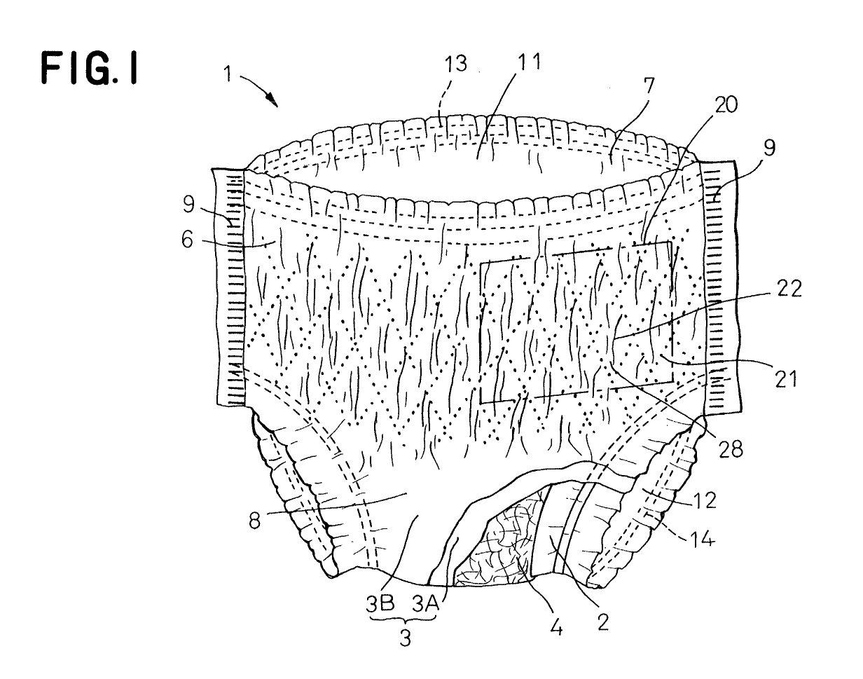

FIG. 1 shows a pull-on or pants type diaper 1 in a perspective view as partially broken away. The diaper 1 is shown in FIG. 1 as contracting transversely of the diaper 1. The diaper 1 comprises a liquid-permeable topsheet 2 intended to contact the wearer's skin, a backsheet 3 intended to contact no the wearer's skin and a liquid-absorbent core 4 disposed between these two sheets 2, 3. The topsheet 2 and the backsheet 3 are put one upon another and bonded to each other by means of hot melt adhesive (not shown) over their portions extending outward beyond a peripheral edge of the absorbent core 4 with which a laminate is formed. This laminate defines a front waist region 6, a rear waist region 7 and a crotch region 8 extending between these two waist regions 6, 7. The front and rear waist regions 6, 7 are put flat and joined together at a plurality of intermittent spots arranged in the vertical direction of the diaper along their transversely opposite side edges so as to define a waist-opening 11 and a pair of leg-openings 12. These openings 11, 12 are provided with circumferentially extending elastic members 13 and 14, respectively, which are disposed between the topsheet 2 and the backsheet 3. These elastic members 13, 14 are secured with appropriate tensions to inner surface(s) of the topsheet 2 and/or the backsheet 3. The backsheet 3 consists of an elastic inner layer 3A being stretchable and contractable and an inelastic outer layer 3B. Of the front and rear waist regions 6, 7 and the crotch region 8, at least the front waist region 6 has a plurality of spots 21 at which the elastic inner layer 3A and the inelastic outer layer are joined to each other and a plurality of pleats extending vertically of the diaper 1.

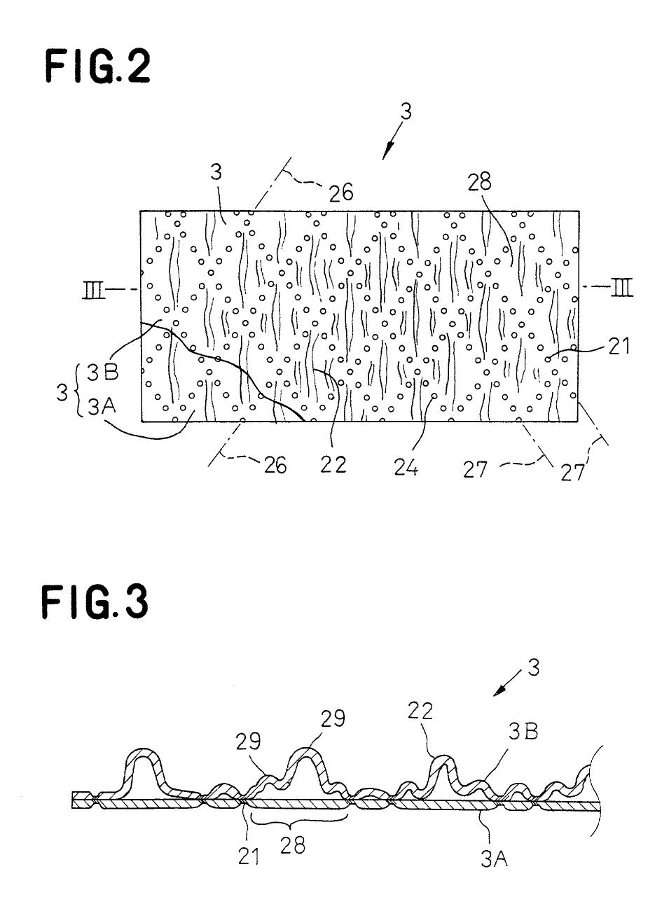

FIG. 2 is a fragmentary plan view showing a portion of the backsheet 3 enclosed by an imaginary line 20 in FIG. 1 and FIG. 3 is a sectional view taken along a line III-III in FIG. 2. The elastic inner layer 3A is made of at least transversely stretchable and contractable material such as elastomer film of thermoplastic synthetic resin or rubber. FIGS. 2 and 3 show the elastic inner layer 3A in its contracted state. The inelastic outer layer 3B is made of substantially unstretchable and uncontactable fibrous material such as inelastic spun bond nonwoven fabric containing thermoplastic synthetic fibers and has a plurality of pleats 22 extending substantially in the vertical direction of the diaper 1. The elastic inner layer 3A and the inelastic outer layer 3B are unitized together by the spots 21 for joining at which a portion of synthetic resin contained in at least one of these layer 3A, 3B is molten and then solidified. The spots 21 for joining are arranged closely along imaginary lines extending in predeterminded directions. Referring to FIG. 2, the spots 21 are arranged along a plurality of parallel imaginary lines 26 obliquely rightward ascending and a plurality of parallel imaginary lines 27 obliquely rightward descending. Each pair of adjacent lines 26 and each pair of adjacent lines 27 cross each other to define each lozenge area 28. In each of the areas 28, the inelastic outer layer 3B floats off from the elastic inner layer 3A so as to form the previously mentioned pleats 22. While both the configuration and the number of the pleats 22 are indeterminate, the pleats 22 may have one or more crest(s) 29 within each of the areas 28, as seen in FIG. 3 showing the pleats 22 in a sectional view.

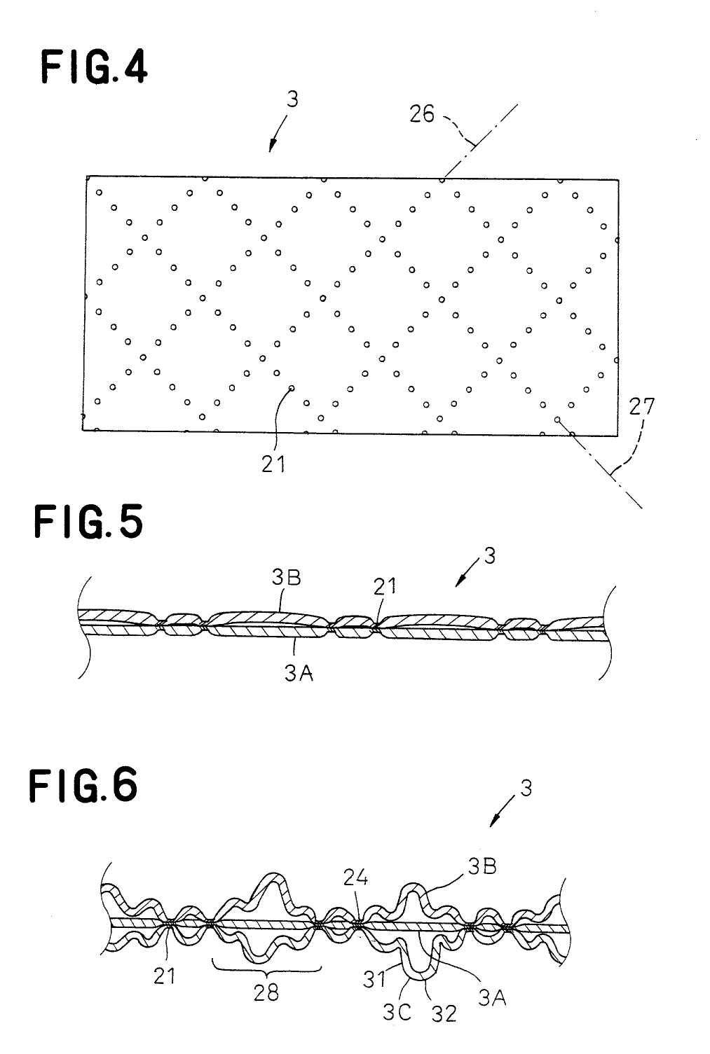

FIGS. 4 and 5 show the backsheet 3 being transversely stretched in a plan view and in a sectional view, respectively. The backsheet 3 is stretched as the elastic inner layer 3A is elastically stretched until the pleats 22 of the inelastic outer layer 3B are completely unfolded. Thereupon, the backsheet 3 becomes substantially flat as shown and the diaper 1 itself shown in FIG. 1 as transversely contracted becomes also flat. Referring to FIG. 4, the lines 26, 27 described by the spots 21 for joining, i.e., the lines 26, 27 along which the spots 21 are arranged are inclined at angle of approximately 45° with respect to the horizontal direction. The illustrated embodiment of the backsheet 3 may be obtained in a manner as illustrated by FIGS. 4 and 5. Specifically, an original web of the inner layer 3A which is elastic for stretch and contraction is stretched at a predeterminded elongation percentage. Then, an original web of the inelastic layer 3B is placed upon the original web of the inner layer 3A. The original webs of these layers 3A, 3B thus placed one upon another are fed between a pair of thermally embossing rolls to obtain a laminated sheet having the spots 21 for joining of a predeterminded pattern. The backsheet 3 may be cut off from this laminated sheet. The contraction of the elastic inner layer 3A causes the pleats 22 to be formed in the direction which is orthogonal to the direction in which the elastic inner layer 3A is contracted. It should be understood that a pattern of the pleats depends on the pattern of the spots 21 for joining.

With the diaper 1 having the backsheet 3 constructed as has been described above, a comfortable soft touch peculiar to the nonwoven fabric can be maintained in spite of the laminate structure of the backsheet 3 with the plastic or rubber film. It is for the reason that the inelastic outer layer 3B floats off from the elastic inner layer 3A in each of the areas 28 so as to form the pleats 22.

FIG. 6 is a view similar to FIG. 3 showing a variant of the backsheet 3 used for the invention. This embodiment of the backsheet 3 comprises, in addition to the elastic inner layer 3A and the inelastic outer layer 3B, an inelastic third layer 3C. The inelastic third layer 3C is made of material similar to the fibrous material of the inelastic outer layer 3B, for example, of spun bond nonwoven fabric containing thermoplastic synthetic fibers. The inelastic third layer 3C is joined to the elastic inner layer 3A at the spots 21 at which the inner and outer layers 3A, 3B are joined to each other. In each of the areas 28, the inelastic third layer 3C floats off from the elastic inner layer 3A in the direction opposite to the inelastic outer layer (downward as viewed in FIG. 6) so as to form pleats 31 having a crest 32. An apparent thickness corresponding to a dimension between the crests 29, 32 presented by the backsheet 3 of such structure in its contracted state is substantially larger than that presented by the backsheet 3 in its stretched state. Such backsheet 3 is comfortably soft and has a high cushioning effect.

To implement the invention, the elastic inner layer 3A forming a constituent of the backsheet 3 is preferably made of elastic film having an elastic recovery of 60˜100% after stretched by 30˜100% and a thickness of 0.01˜0.1 mm. The elastic film is preferably used with an elastically deformable tension. The inelastic outer layer 3B and the inelastic third layer 3C are preferably made of nonwoven fabric having a basis density of 15˜200 g/m². Size and pattern of the spots 21 for joining may be selected so that they do not substantially affect the elasticity of the elastic inner layer 3A and can form the pleats 22 providing the diaper 1 with a desired softness. This is achieved, for example, by the spots 21 each having an area of 0.7˜9 mm² and spaced apart from the adjacent spot 21 by 1˜5 mm. The lines 26, 27 described by the spots 21 successively adjacent one to another may be either straight or curved. In the case of the embodiment shown by FIG. 4, the spots 21 each defined by a circle having a diameter of approximately 1.8 mm are spaced apart one from another by a center-to-center distance of approximately 4 mm so as to form the straight lines 26, 27, respectively. Both each pair of adjacent straight lines 26 and each pair of adjacent straight lines 27 are spaced apart in parallel with each other by approximately 20 mm, respectively. The spots at which the inelastic outer layer 3B and the inelastic third layer 3C are joined to the elastic inner layer 3A, respectively, may be separately provided instead of providing them commonly for the inelastic outer layer 3B and the inelastic third layer 3C as shown. Concerning the elastic inner layer 3A, it is not essential for the elastic inner layer 3A to cover the diaper 1 entirely. It is also possible to provide it on a part of the diaper 1, for example, along transversely opposite side edges of the front waist region 6 and/or the rear waist region 7 so as to form the pleats 22 only these regions of the diaper 1.

The entire disclosure of Japanese Patent Application No. Hei8-259571 filed on Sep. 30, 1996 including specification, claims, drawings and abstract are incorporated herein by reference in its entirety.

Having described our invention as related to the embodiments shown in the accompanying drawings, it is our intention that the invention be not limited by any of the details of description, unless otherwise specified, but rather be construed broadly within its spirit and scope as set out in the accompanying claims.

What is claimed is:

1. A disposable diaper having a front waist region, a rear waist region and a crotch region therebetween, said diaper comprising a liquid-permeable topsheet, a liquid-impermeable backsheet and a liquid-absorbent core disposed therebetween, wherein:
said backsheet in at least said front waist region comprises an elastic inner layer and an inelastic outer layer overlapped with said elastic inner layer; and
said elastic inner layer and said inelastic outer layer fabric are joined to each other at a plurality of intermittently arranged spots, with said elastic inner layer being stretched transversely of said diaper so that, in areas enclosed by intersecting lines each described by a plurality of said spots closely arranged in succession, said inelastic outer layer is free to float off from said elastic inner layer and thereupon to form a plurality of pleats as said elastic inner layer is made free to contract transversely of said diaper.

2. The diaper according to claim 1, wherein an inelastic third layer made of inelastic fibrous material is joined to said elastic inner layer at a plurality of intermittently arranged spots for joining so that said inelastic third layer may also float off from said elastic inner layer and form a plurality of pleats as said elastic inner layer is made free to contract.

3. The diaper according to claim 1, wherein at least one of said elastic inner layer and said inelastic outer layer contains thermally meltable synthetic resin and said spots for joining at which said elastic inner layer and said inelastic outer layer are joined to each other are formed by said resin molten and then solidified.

4. The diaper according to claim 2, wherein at least one of said elastic inner layer, said inelastic outer layer and said inelastic third layer contains thermally meltable synthetic resin and said spot for joining at which said elastic inner layer and said two inelastic outer and third layers are joined one to another are formed by said resin molten and then solidified.

5. The diaper according to claim 2, wherein said inelastic outer layer is joined to an outer surface of said elastic inner layer and said third inelastic layer is joined to an inner surface of said elastic inner layer at the common spots for joining.

6. The diaper according claim 1, wherein said elastic inner layer is made of at least transversely stretchable and contractable material and said inelastic outer and third layers are made of substantially unstretchable and uncontractable fibrous materials, respectively.

7. The diaper according claim 1, wherein the material of said elastic inner layer is plastic or rubber film and the materials of said inelastic outer and third layers are nonwoven fabrics, respectively.
